(19) United States
(12) Patent Application Publication
(10) Pub. No.: US 2002/0081671 A1
(43) Pub. Date: Jun. 27, 2002

(54) Reductive amination of alpha-ketodicarboxylic acid derivatives

(75) Inventors: Kai Rossen, Hanau (DE); Martin Sarich, Alzenau (DE); Milan Latinovic, Nidda (DE); Claudia Rollmann, Alzenau (DE); Andreas Bommarius, Atlanta, GA (US)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: Degussa AG, Duesseldorf (DE)

(21) Appl. No.: 09/985,475

(22) Filed: Nov. 5, 2001

(30) Foreign Application Priority Data
Nov. 3, 2000 (DE) 100 54 492.4

(51) Int. Cl. C12P 13/04; C12P 13/22; C12P 13/06

(52) U.S. Cl. 435/106; 435/108; 435/116

(57) ABSTRACT

The invention relates to an enzymatic method for reductive amination of α-ketodicarboxylic acid derivatives or salts thereof using an amino acid dehydrogenase.

### CROSS-REFERENCE TO RELATED APPLICATIONS

The present application claims priority to German Application No. DE 10054492.4 filed Nov. 3, 2000; the entire contents of which are incorporated herein by reference.

### BACKGROUND OF THE INVENTION

1. Field of the Invention

The present invention relates to an enzymatic method for reductive amination of α-ketodicarboxylic acid derivatives of general formula (I) or salts thereof.

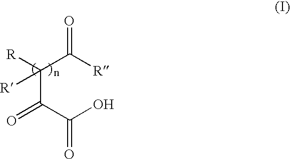

2. Discussion of the Background

Reductive amination of α-ketodicarboxylic acid derivatives yield α-aminodicarboxylic acid derivatives that have particularly high enantiomeric concentrations. These products are of particular importance as they may be used as precursors in the synthesis of biologically active molecules, especially pharmaceuticals (N. Moss et al., Synthesis 1977, 32 ff.).

α-Aminodicarboxylic acid derivatives may be obtained by classical chemical synthesis. However, because these compounds are important, attempts are constantly being made to improve the preparation process emphasizing high yield and high purity on an industrial scale. The classical chemical approach requires the use of toxic metals (e.g., lead) or organometallic reagents at low temperatures (J. Org. Chem. 1999, 64, 4362 ff.; J. Org. Chem. 1990, 55, 3068 ff.). Therefore, using these reagents renders the classical approach unfavorable for industrial scale preparation of α-aminodicarboxylic acid derivatives.

It has been previously described that α-ketocarboxylic acids may be subjected to enzymatic reductive amination by using an amino acid dehydrogenase as a biocatalyst. However, these reactions have only limited success and are conducted on a small scale (J. Biotechn. 1997, 53, 29; J. Org. Chem. 1990, 55, 5567; Enzyme Catalysis in Organic Synthesis, Eds.: K. Drauz and H. Waldmann, VCH, 1995, 633 ff.). These studies show that the preferred substrates of leucine dehydrogenase (LeuDH) are relatively small aliphatically substituted α-keto acids. In contrast, phenylalanine dehydrogenase (PheDH) reacts more readily with substrates having very bulky hydrophobic substituents in the α-position (J. Biotechn. 1997, 53, 29).

However, there remains a critical need for improved methods of producing α-aminodicarboxylic acid derivatives from α-ketodicarboxylic acid derivatives of general formula (I) to serve as precursors in the synthesis of pharmaceuticals. On a commercial or industrial scale even small improvements in the yield, purity, or efficiency of production of α-aminodicarboxylic acid derivatives from α-ketodicarboxylic acid derivatives of general formula (I) are economically significant. Prior to the present invention, it was not recognized that amino acid dehydrogenases would provide such an advantage on an industrial scale.

### SUMMARY OF THE INVENTION

An object of the present invention is to provide a method by which α-ketodicarboxylic acid derivatives, which have a tertiary carbon atom in the position adjacent to the keto group, can be reductively aminated by enzymatic means. Such a method would provide an ecological and economic advantage to the industrial synthesis of α-aminodicarboxylic acid derivatives.

A further object of the invention is provide α-aminodicarboxylic acid derivatives suitable for the use in the synthesis of biologically active principles, specifically pharmaceuticals.

The above objects highlight certain aspects of the invention. Additional objects, aspects and embodiments of the invention are found in the following detailed description of the invention.

### DETAILED DESCRIPTION OF THE INVENTION

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and are not intended to be limiting.

The invention provides a method of producing α-aminodicarboxylic acid derivatives by contacting α-ketodicarboxylic acid derivatives of general formula (I) or of salts thereof with an amino acid dehydrogenase.

High concentrations of enantiomeric forms of α-ketodicarboxylic acid derivatives of general formula (I) or of salts thereof can be obtained through the use of an amino acid dehydrogenase for reductive amination:

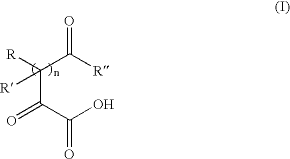

wherein

n=1 to 3,

R, R′ independently of one another denote H, (C₁-C₈)-alkyl, (C₁-C₈)-alkoxy, (C₂C₈)-alkoxyalkyl, (C₆-C₁₈)-aryl, (C₇-C₁₉)-aralkyl, (C₃-C₁₈)-heteroaryl, (C₄-C₁₉)-heteroaralkyl, (C₁-C₈)-alkyl-(C₆-C₁₈)-aryl, (C₁-C₈)-alkyl-(C₃-C₁₈)-heteroaryl, (C₃-C₈)-cycloalkyl, (C₁-C₈)-alkyl-(C₃-C₈)-cycloalkyl, (C₃-C₈)-cycloalkyl-(C₁-C₈)-alkyl,

or R and R′ together form a ring via a (C₂-C₅)-alkylene bridge, which can contain one or more double bonds and/or can be substituted with one or more (C₁-C₈)-alkyl, (C₁-C₈)-acyl, (C₁-C₈)-alkoxy or (C₂-C₈)-alkoxyalkyl and/or can contain hetero atoms such as N, O, P, S in the ring, with the proviso that the R and R′ groups bonded to different C atoms are independent of one another and that R, R′ in neighboring position to the keto function cannot be H, R″ denotes OR, NHR, NRR′, wherein in these cases R or R′ cannot be (C₁-C₈)-alkoxy,

Preferably, α-ketodicarboxylic acid derivatives of general formula (I) or salts thereof of the present invention are compounds in which

n=1 or

R, R′ is (C₁-C₈)-alkyl or

R and R′ together form a ring via a (C₂-C₅)-alkylene bridge or

R″ is OH, NH₂.

The amino acid dehydrogenase is preferably a leucine dehydrogenase (LeuDH) or phenylalanine dehydrogenase (PheDH). It has been previously described that LeuDH or PheDH prefer substrates that have either small aliphatic or very bulky hydrophobic α-substituents, respectively. Therefore, it is unexpected that these enzymes would admit hydrophilic groups such as acids and their salts, esters or amides.

PheDH maybe obtained from the organisms cited in U.S. Pat. No. 5,851,810, J. Biotechn. 1997, 53, 29 and J. Org. Chem. 1990, 55, 5567. More preferred is the PheDH from Rhodococcus M4 (DSM3041; U.S. Pat. No. 5,416,019; seq. 2). However, the skilled artisan will recognize that other amino acid dehydrogenases can be employed. Any amino acid dehydrogenase substitution must account for optimization of product conversion, stability, and availability.

The amino acid dehydrogenase enzymes can be used in free form as homogeneously purified enzymes or as enzymes synthesized by recombinant techniques. Furthermore, the enzymes can also be used as a constituent of an intact (guest) organism or in combination with the digested cell mass, which can be purified to any degree desired, of the respective host organism. It is also possible to use the enzymes in an immobilized form (Bhavender P. Sharma, Lorraine F. Bailey and Ralph A. Messing, “Immobilized biomaterials—Techniques and applications”, Angew. Chem. 1982, 94, 836-852.). It is preferred that immobilization be achieved by lyophilization (Dordick et al., J. Am. Chem. Soc. 194, 116, 5009-5010; Okahata et al., Tetrahedron Lett. 1997, 38, 1971-1974; Adlercreutz et al., Biocatalysis 1992, 6, 291-305). Most particularly preferred is lyophilization in the presence of surfactant substances, such as Aerosol OT or polyvinylpyrrolidone or polyethylene glycol (PEG) or Brij 52 (diethylene glycol monocetyl ether) (Goto et al., Biotechnol. Techniques 1997, 11, 375-378). Use as CLECs is also conceivable (Vaghjiani et al., Biocat. Biotransform. 2000, 18, 157 ff.).

Amino acid dehydrogenases, especially leucine dehydrogenases and phenylalanine dehydrogenages, are generally coenzyme-dependent (NADH/NADPH) enzymes (Beyer, Walter, Textbook of Organic Chemistry, 22nd Edition, S. Hirzel Verlag, Stuttgart, p. 886). On an industrial scale, it may be advantageous to regenerate the coenzyme (Enzyme Catalysis in Organic Synthesis, Eds.: K. Drauz and H. Waldmann, VCH, 1995, 596 ff.), especially by means of a formate dehydrogenase (FDH) and a formate source, in order to reduce the feed quantity of NADH/NADPH required (J. Biotechn. 1997, 53, 29).

The source of formate for the inventive reaction may be supplied by substances familiar to the skilled artisan (Enzyme Catalysis in Organic Synthesis, Eds.: K. Drauz and H. Waldmann, VCH, 1995, p. 596). However, the preferred formate source of the present invention is the salt of formic acid and, most preferably, ammonium formate.

It is possible to use many forms of FDH that may be freely selected by the skilled artisan and such forms have been described previously (German Patent Application 19753350.7). The FDH from C. boidinii is preferred and more preferably a FDH form that has been stablized by mutation.

Preferred quantities of feed substances and enzymes for use in the present reaction are shown in the table below. These ranges correspond to reaction optimums and ranges that are economically reasonable.

TABLE
Quantities of feed substances and enzymes
(relative to 1 g of the compound of general formula (I)).

| Feed | | | | | | |
|---|---|---|---|---|---|---|
| substance/enzyme | Min. value | | Max. value | | Preferred range | |
| Formate | 0.1 | mol/l | 3.0 | mol/l | 1 to 2.0 | mol/l |
| FDH | 2 | U | 50 | U | 10 to 30 | U |
| NADH/NADPH | 1 | mg | 1 | g | 2 to 20 | mg |
| PheDH | 1 | U | 100 | U | 5 to 25 | u |

The preferred concentration of the compound of general formula (I) to be used in the present invention is 0.05 mol/l to 3.0 mol/l, preferably 0.5 mol/l to 1.5 mol/l. From an ecological viewpoint, this is advantageously carried out in water as the solvent. Addition of water-soluble organic solvents may be necessary for solubility reasons. In this case preferably methanol, ethanol, acetone, glacial acetic acid (advantageously up to 10%) is then added to the reaction mixture.

The pH of the reaction should be maintained at a value of 7.5 to 10.0, preferably from 8.0 to 9.0. Most particularly preferably, a pH of 8.4 is used.

The reductive amination reaction of the present invention should by kept at a temperature that would maintain the functional capability of the enzymes. Further, the temperature must not be too low, since the reaction would otherwise take place too slowly. The preferred temperature range for the inventive reaction is from 15° to 50° C., most preferably between 30° and 40° C.

In an especially preferred embodiment, the inventive process takes place in an enzyme membrane reactor (described in German Patent Application 19910691.6).

A further object of the invention relates to the use of the compounds synthesized according to the reductive amination reaction of the present invention in a method for synthesis of biologically active principles, preferably pharmaceuticals.

The α-ketodicarboxylic acid derivatives of general formula (I) may be synthesized by a method known to the skilled artisan (J. Biotech. 1997, 53, 29; J. Org. Chem. 1990, 55, 5567). In general, however, the α-ketodicarboxylic acid derivatives can be obtained by reacting alkylcarboxylic acid derivatives with compounds capable of electrophilic substitution, such as haloorganic compounds (RHal, R′Hal), in the presence of a base in an inert organic solvent. Thereafter the terminal methyl function is oxidized to the acid.

The resultant α-ketodicarboxylic acid derivatives are then brought into contact with the dehydrogenase enzymes in an aqueous medium in the presence of a cofactor regeneration system. The reaction in question usually takes pace quantitatively and with high chiral induction as shown below:

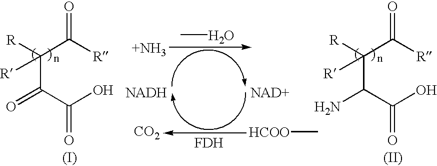

The resultant amino acids can be purified by the methods known to the skilled artisan. Preferably the amino acids are isolated by ultrafiltration followed by ion-exchange chromatography or crystallization (Houben-Weyl, Volume E16d, Georg Thieme Verlag, Stuttgart, 1992, pp. 406 ff.).

Examples of (C₁-C₈)-alkyl groups are methyl, ethyl, n-propyl, isopropyl, n-butyl, isobutyl, sec-butyl, tert-butyl, pentyl, hexyl, heptyl or octyl as well as all of their bond isomers. The (C₁-C₈)-alkoxy group corresponds to the (C₁-C₈)-alkyl group, with the proviso that it is bonded to the molecule via an oxygen atom. The (C₂-C₈)-alkoxyalkyl group is to be understood as groups in which the alkyl chain is interrupted by at least one oxygen functions although two oxygen atoms cannot be joined to one another. The number of carbon atoms indicates the total number of carbon atoms contained in the group. A (C₂-C₅)-alkylene bridge is a carbon chain containing two to five C-atoms, this chain being bonded to the molecule in question via two of its C-atoms, which must be different.

The groups just described can be substituted with five or more halogen atoms and/or with one or more groups that contain N, O, P, S, Si atoms. These are in particular alkyl groups of the foregoing type, which contain one or more of these hetero atoms in their chain or which are bonded to the molecule via one of these hetero atoms.

By (C₃-C₈)-cycloalkyl there are understood cyclopropyl, cyclobutyl, cyclopentyl, cyclohexyl and cycloheptyl groups, etc. These can be substituted with one or more halogen atoms and/or groups containing N, O, P, S, Si atoms, and/or can contain N, O, P, S atoms in the ring, examples of these are 1-, 2-, 3-, 4-piperidyl, 1-, 2-, 3-pyrrolidinyl, 2-, 3-tetrahydrofuryl, 2-, 3-, 4-morpholinyl.

A (C₃-C₈)-cycloalkyl-(C₁-C₈)-alkyl group denotes a cycloalkyl group such as described hereinabove, which is bound to the molecule via an alkyl group such as specified hereinabove.

Within the scope of the invention, (C₁-C₈)-acyloxy denotes an alkyl group such as defined hereinabove with at most 8 C-atoms, which is bound to the molecule via a COO function.

Within the scope of the invention, (C₁-C₈)-acyl denotes an alkyl group such as defined hereinabove with at most 8 C-atoms, which is bound to the molecule via a CO function.

By a (C₆-C₁₈)-aryl group there is understood an aromatic group with 6 to 18 C atoms. Particular examples thereof are compounds such as phenyl, naphthyl, anthryl, phenanthryl, biphenyl groups or systems of such type annelated to the molecule in question, examples being indenyl systems, which may or may not be substituted with (C₁-C₈)-alkyl, (C₁-C₈)-alkoxy, N(C₁-C₈)-alkyl, (C₁-C₈)-aryl, (C₁-C₈)-acyloxy.

A (C₇-C₁₉)-aralkyl group is a (C₆-C₁₈)-aryl group bonded to the molecule via a (C₁-C₈)-alkyl group.

Within the scope of the invention, a (C₃-C,₁₈)-heteroaryl group designates a five-membered, six-membered or seven-membered aromatic ring system comprising 3 to 18 C atoms and containing hetero atoms such as nitrogen, oxygen or sulfur in the ring. In particular, groups such as 1-, 2-, 3-furyl, 1-, 2-, 3-pyrrolyl, 1-, 2-, 3-thienyl, 2-, 3-, 4-pyridyl, 2-, 3-, 4-, 5-, 6-, 7-indolyl, 3-, 4-, 5-pyrazolyl, 2-, 4-, 5-imidazolyl, acridinyl, quinolinyl, phenanthridinyl, 2-, 4-, 5-, 6-pyrimidinyl are regarded as such hetero atoms.

By (C₄-C₁₉)-heteroalkyl there is understood a heteroaromatic system corresponding to the (C₇-C₁₉)-aralkyl group.

As halogens (Hal), fluorine, chlorine, bromine and iodine may be used.

Having generally described this invention, a further understanding can be obtained by reference to certain specific examples which are provided herein for purposes of illustration only, and are not intended to be limiting unless otherwise specified.

### EXAMPLE

Preparation of 1-acetylcyclopropanecarboxylic acid ethyl ester

| | | | | |
|---|---|---|---|---|
| 215 | mmol | ethyl acetoacetate | 28.55 | g |
| 250 | mmol | dibromomethane | 49.34 | g |
| 1.075 | mol | potassium carbonate | 150 | g |
| 650 | ml | dimethyl sulfoxide | 650 | ml |

Potassium carbonate was added to a solution of ethyl acetoacetate and dibromomethane in dimethyl sulfoxide (DMSO). The suspension was stirred for 18 hours at 25° C. After addition of 550 ml of water, the product was extracted in 300 ml of methyl tert-butyl ether (MTBE). After two aqueous extractions of the organic phase, the product-containing phase was concentrated by evaporation.

The raw product was then dissolved in 100 ml of water and 100 ml of methanol and, after addition of 8 g of NaOH, was saponified overnight to the carboxylic acid. The solution obtained after evaporation of the methanol was used directly in the subsequent oxidation step.

| | | | |
|---|---|---|---|
| 400 | mmol | KMnO₄ | 63 g |
| 400 | mmol | NaOH | 16 g |
| 300 | ml | water | |

The aqueous solution of the methylketane was added to a solution of KMnO₄ and NaOH in H₂O while cooling with ice. The solution was then stirred overnight at 25° C. The precipitated manganese dioxide was filtered off and the resulting solution was used directly for the enzymatic reductive amination (yield 75% by HPLC).

Reductive amination of keto-cyclopropyl aspartate with PheDH/FDH

| | |
|---|---|
| Keto-cyclopropyl aspartate (KS) | 0.5 M |
| Ammonium formate | 1.5 M |
| NAD⁺ trihydrate | 5.6 mg per g of KS |
| phenylalanine dehydrogenase (PheDH) | 6.75 U per g of KS |
| formate dehydrogenase (FDH) | 11.1 U per g of KS |
| pH | 8.2 to 8.5 |
| Temperature | 30° C. |
| Reaction time | 20 to 30 hours |

The keto-cyclopropyl aspartate was dissolved along with ammonium formate in demineralized water while stirring. The temperature and pH were adjusted as indicated above. Subsequently, PheDH, FDH and NAD⁺ trihydrate were added, while the temperature and pH were kept constant throughout the entire reaction. If it becomes necessary, the pH may be restored by addition of ammonia solution, formic acid, or by dilution.

Upon completion of the reaction, the enzymes (PheDH and FDH) were separated by ultrafiltration. The amino acid was subsequently isolated in manner known to the skilled artisan by ion-exchange chromatography (yield 99%).

The spectra were recorded on a DRX 500 MHz NMR spectrometer of the Bruker Co. (Rheinstetten, Germany) at a frequency of 500.13 MHz for protons. The chemical shifts were measured relative to tetramethylsilane (TMS) as the internal standard. The measurements were performed in DMSO-d₆ at 303 K.

¹H NMR δ 1.20 (m, 1 H), 1.25 (m, 2 H), 1.34 (m, 1 H), 3.56 (s, 1 H), 8.37 (b, 3 H).

1. A method for the reductive amination of α-ketodicarboxylic acid derivatives of general formula (I) or of salts thereof 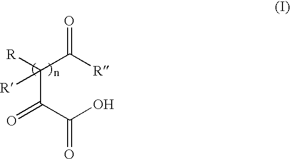
wherein
n=1 to 3,
R, R′ independently of one another denote H, (C₁-C₈)-alkyl, (C₁-C₈)-alkoxy, (C₂-C₈)-alkoxyalkyl, (C₆-C₁₈)-aryl, (C₇-C₁₉)-aralkyl, (C₃-C₁₈)-heteroaryl, (C₄-C₁₉)-heteroaralkyl, (C₁C₈)-alkyl-(C₆-C₁₈)-aryl, (C₁-C₈)-alkyl-(C₃-C₁₈)-heteroaryl, (C₃-C₈)-cycloalkyl, (C₁-C₈)-alkyl-(C₃-C₈)-cycloalkyl, (C₃-C₈)-cycloalkyl-(C₁-C₈)-alkyl,
or R and R′ together form a ring via a (C₂-C₅)-alkylene bridge, which can contain one or more double bonds and/or can be substituted with one or more (C₁-C₈)-alkyl, (C₁-C₈)-acyl, (C₁-C₈)-alkoxy or (C₂-C₈)-alkoxyalkyl and/or can contain hetero atoms such as N, O, P, S in the ring,
with the proviso that the R and R′ groups bonded to different C atoms are independent of one another and that R, R′ in neighboring position to the keto function are not H,
R″ denotes OR, NHR, NRR′, wherein in these cases R or R′ are not (C₁-C₈)-alkoxy,
comprising contacting said α-ketodicarboxylic acid derivatives with an amino acid dehydrogenase.

2. The method according to claim 1, wherein
n=1 or
R, R′ is (C₁-C₈)-alkyl or
R and R′ together form a ring via a (C₂-C₅)-alkylene bridge or
R″ is OH, NH₂.

3. The method according to claim 1, wherein the compound of general formula (I) is in a concentration of from 0.05 mol/l to 3.0 mol/l.

4. The method according to claim 1, wherein the compound of general formula (I) is in a concentration of from 0.5 mol/l to 1.5 mol/l.

5. The method according to claim 1, wherein said amino acid dehydrogenase is a leucine dehydrogenase or phenylalanine dehydrogenase.

6. The method according to claim 1, wherein said amino acid dehydrogenase is in an amount of from 1 U to 100 U.

7. The method according to claim 1, wherein said amino acid dehydrogenase is in an amount of from 5U to 25 U.

8. The method according to claim 1, wherein the amino acid dehydrogenase is in a form selected from the group consisting of homogenously pure, a component of a cell extract, and an immobilized protein.

9. The method according to claim 1, wherein said contacting is performed in an aqueous medium.

10. The method according to claim 9, wherein the pH of the aqueous medium is maintained from 7.5 to 10.

11. The method according to claim 9, wherein the pH of the aqueous medium is maintained from 8 to 9.

12. The method according to claim 9, wherein the pH of the aqueous medium is maintained at 8.4.

13. The method according to claim 9, wherein said aqueous medium further comprises a water-soluble organic solvent selected from the group consisting of methanol, ethanol, acetone, and glacial acetic acid.

14. The method according to claim 1, wherein said contacting is performed in the presence of a coenzyme.

15. The method according to claim 14, wherein said coenzyme is NADH or NADPH.

16. The method according to claim 14, wherein said coenzyme is in an amount of from 1 mg to 1 g.

17. The method according to claim 14, wherein said coenzyme is in an amount of from 2 mg to 20 mg.

18. The method according to claim 14, further comprising regenerating the coenzyme.

19. The method according to claim 18, wherein said regenerating comprises contacting said coenzyme with a formate dehydrogenase.

20. The method according to claim 19, wherein said contacting is performed in the presence of formate.

21. The method according to claim 20, wherein formate is in the concentration of from 0.1 mol/l to 3.0 mol/l.

22. The method according to claim 20, wherein formate is in the concentration of from 1 mol/l to 2.0 mol/l.

23. The method according to claim 1, wherein the reaction temperature is from 15° to 50° C.

24. The method according to claim 1, wherein the reaction temperature is from 30° to 40° C.

25. The method according to claim 1, wherein said contacting is performed in an enzyme membrane reactor.

26. The method according to claim 1, further comprising purifying the products.

27. The method according to claim 26, wherein said purifying is performed by ultrafiltration followed by ion-exchange chromatography.

28. The method according to claim 26, wherein said purifying is performed by ultrafiltration followed by crystallization.
